(19) United States
(12) Patent Application Publication
(10) Pub. No.: US 2011/0319603 A1
(43) Pub. Date: Dec. 29, 2011

(54) METHOD FOR FLUORESCENTLY LABELING PROTEIN

(75) Inventors: Kazuya Kikuchi, Osaka (JP); Yuichirou Hori, Osaka (JP); Hideki Ueno, Osaka (JP)

(73) Assignee: OSAKA UNIVERSITY, Suita-shi, Osaka (JP)

(21) Appl. No.: 13/255,431

(22) PCT Filed: Mar. 10, 2010
(86) PCT No.: PCT/JP2010/054024

§ 371 Date: Sep. 8, 2011

(30) Foreign Application Priority Data
Mar. 10, 2009 (JP) 2009-056305

(51) Int. Cl. C07K 14/00; C12N 15/63; C07C 245/04; C07D 249/04

(52) U.S. Cl. 530/409; 548/255; 435/320.1; 534/551

(57) ABSTRACT

A method for fluorescently labeling a protein is provided that can also be used for labeling a biomolecule (a protein) in vivo with fluorescence. The method for fluorescently labeling a protein includes obtaining a fusion protein of a target protein to be labeled and, for example, a photoactive yellow protein (PYP), and fluorescently labeling the target protein by reacting the fusion protein with a compound represented by Formula (I), wherein in the compound represented by Formula (I), a coumarin skeleton in Formula (A) or a benzene skeleton in Formula (B) is intramolecnlarly associated with a group X and as a result, fluorescence derived from the group X is suppressed and when in the compound of Formula (I), the coumarin skeleton in Formula (A) or the benzene skeleton in Formula (B) is bound to the PYP or the PYP-derived protein contained in the fusion protein, the coumarin skeleton in Formula (A) or the benzene skeleton in Formula (B) is intramolecnlarly dissociated from the group X, and thereby fluorescence derived from the group X can be emitted.

### TECHNICAL FIELD

The present invention relates to a method for fluorescently labeling a protein.

### BACKGROUND ART

Recently, bioimaging for observing the functions of biomolecules such as proteins inside living organisms has been receiving attention. The observation of the biomolecule functions is being conducted widely by labeling biomolecules with fluorescence to visualize the localization or movement of the biomolecules inside living organisms. A general method for labeling biomolecules with fluorescence is one using a genetic engineering procedure to allow a fluorescent protein to express as a fusion protein of a target biomolecule (protein). This fluorescent protein has disadvantages in, for example, its large molecular size and a difficulty in observing near-infrared fluorescence with a high tissue penetration. Therefore, attention is focused on small organic molecules having smaller molecular sizes than that of a fluorescent protein and excellent fluorescent properties. With respect to technologies for labeling target proteins with small organic molecules, there are commercialized labeling kits in which HaloTag (registered trademark) (Promega KK) (see, for example, Non-Patent Document 1) and SNAP-tag (registered trademark) (see, for example, Non-Patent Document 2) are used. These labeling methods each use an enzyme reaction to label target biomolecules with fluorescence. Therefore, the specificity in labeling target biomolecules with small organic molecules is very high but fluorescence derived from the small organic molecules that are not bound to the biomolecules is observed as background signals. In order to reduce the background signals, after being fluorescently labeled with the small organic molecules, washing is necessary to remove unreacted small organic molecules. However, it is generally difficult to wash biomolecules present in a cell or in vivo and it is often not possible to wash them at all. Accordingly, in order to observe the functions of biomolecules in a cell or in vivo, the above-mentioned labeling methods cannot be considered versatile.

### PRIOR ART DOCUMENTS

### Non-Patent Documents

Non-Patent Document 1: Qureshi, M. H. et at, J. Biol. Chem., 2001, 276, p. 46422-8

Non-Patent Document 2: Proc. Natl. Acad. Sci. USA 2004, 101, p. 9955-9959

### DISCLOSURE OF INVENTION

### Problem to be Solved by the Invention

Therefore, the present invention is intended to provide a method for fluorescently labeling a protein, which can be used for labeling a biomolecule (a protein) with fluorescence in a cell or in vivo.

### Means for Solving Problem

The present invention is a method for fluorescently labeling a protein that includes obtaining a fusion protein of a target protein to be labeled and a photoactive yellow protein (PYP) or a PYP-derived protein, and fluorescently labeling the target protein by reacting the fusion protein with a compound represented by Formula (I) below or a salt thereof,

wherein in the compound represented by Formula (I) or the salt thereof a coumarin skeleton in Formula (A) or a benzene skeleton in Formula (B) is intramolecularly associated with a group X and as a result, fluorescence derived from the group X is suppressed, and

when in the compound represented by Formula (I) or a salt thereof the coumarin skeleton in Formula (A) or the benzene skeleton in Formula (B) is bound to the PYP or the PYP-derived protein contained in the fusion protein, the coumarin skeleton in Formula (A) or the benzene skeleton in Formula (B) is intramolecularly dissociated from the group X, and thereby fluorescence derived from the group X can be emitted.

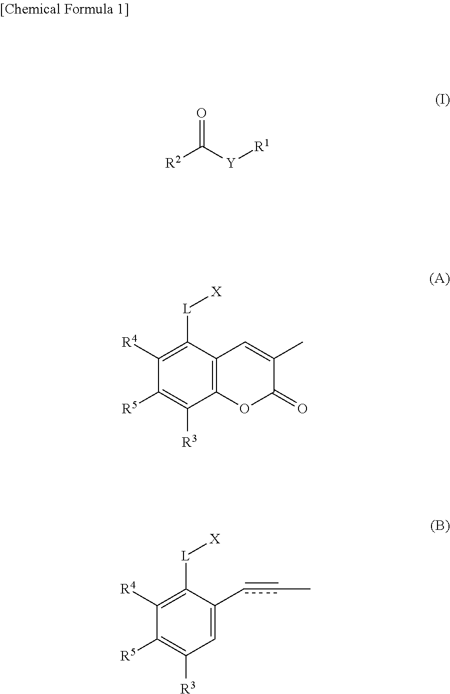

In Formula (I),
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including the coumarin skeleton or a group represented by Formula (B) including the benzene skeleton,
wherein in Formula (A) and Formula (B),
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen,
R⁵ is a hydroxy group, an amino group, an amino group that has been mono- or di-substituted with lower alkyl, or a lower alkoxy group, and

[Chemical Formula 2]

[symbol]

denotes a double bond or a triple bond.

### Effects of the Invention

In the method for fluorescently labeling a protein according to the present invention, high fluorescence is exhibited only when a target protein has been labeled. Therefore, the fluorescence background can be minimized without removing small organic molecules that are not labeling proteins. Accordingly, highly accurate fluorescence imaging can be carried out. Furthermore, since it is not necessary to remove unreacted small organic molecules, the method is suitable for observing biomolecules in vivo.

### BRIEF DESCRIPTION OF DRAWINGS

FIG. 1(a) shows a CBB-stained image obtained after the reaction between a PYP and a compound FCTP and FIG. 1(b) shows a fluorescent image obtained after the reaction between the PYP and the compound FCTP.

FIG. 2(a) shows a CBB-stained image obtained after the reaction between a PYP and a compound FCTP in a cell lysate and FIG. 2(b) shows a fluorescent image obtained after the reaction between the PYP and the compound FCTP in the cell lysate.

FIG. 3(a) shows time-dependent fluorescence spectra of 8 μM of compound FCTP in the presence of 5 μM of PYP.

FIG. 3(b) shows time-dependent excitation spectra of 8 μM of compound FCTP in the presence of 5 μM of PYP.

FIG. 3(c) shows fluorescence spectra (excitation at 500 nm) of 8 μM of compound FCTP.

FIG. 4 shows fluorescence microscopic images illustrating a labeling reaction using a FCTP in a cultured cell.

FIG. 5 shows the procedure of a labeling reaction using a CATP in a cultured cell.

FIG. 6 shows fluorescence microscopic images illustrating a labeling reaction using a CATP in a cultured cell.

### DESCRIPTION OF THE INVENTION

A photoactive yellow protein (PYP) is a photoreceptor protein isolated from purple photosynthetic bacteria, Ectothiorhodospira halophila (Halorhodospira halophila). Since the purple photosynthetic bacteria are eubacteria, a PYP-derived endogenous protein is unlikely to adversely affect fluorescence labeling when a PYP is used in a cell. Furthermore, the PYP consists of 125 amino acid residues (SEQ ID NO: 1 of the sequence listing) and is a relatively small protein (14 kDa). In the PYP, a chromophore, p-coumaric acid, binds to the 69th cysteine residue through a thioester bond. It is known that various analogs other than p-coumaric acid each are bound to a PYP (see, for instance, compounds shown in Scheme 1 below as well as, for example, Cordgunke et al., Proc. Natl. Acad. Sci, USA, 1998, 95, pp 7396-7401 and Koon et al., The Journal of Biological Chemistry 1996, 271, pp. 31949-31956).

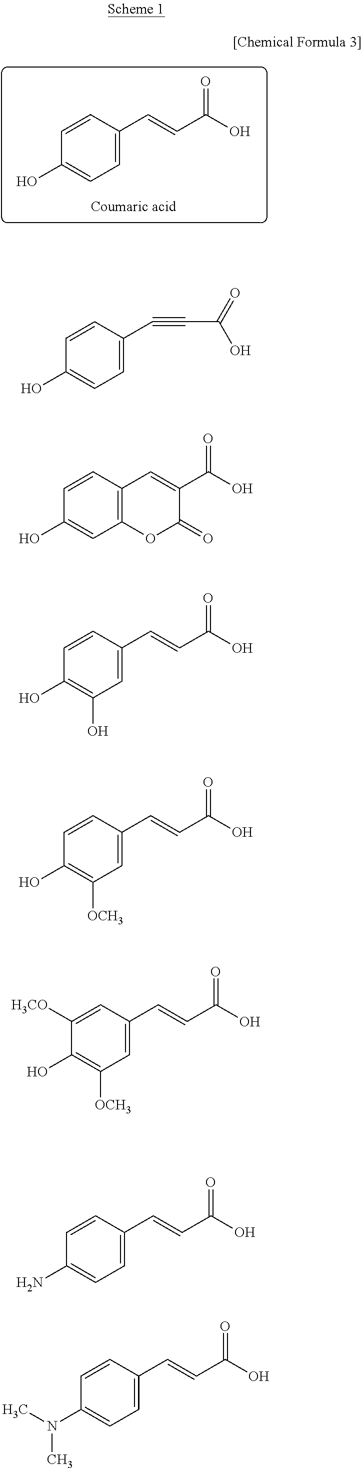

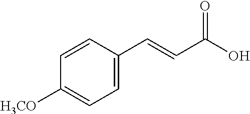

According to the labeling method of the present invention, in a compound represented by Formula (I) or a salt thereof the coumarin skeleton in Formula (A) or the benzene skeleton in Formula (B) and the group X, which are bound to each other through the linker L, are intramolecularly associated and as a result, the fluorescence derived from the group X is suppressed (see Scheme 2). In the labeling method of the present invention, a fusion protein obtained by fusing a PYP or PYP-derived protein with a target protein to be labeled is reacted with the compound of Formula (I) or the salt thereof. With respect to the PYP or PYP-derived protein of the fusion protein, when the coumarin skeleton in Formula (A) or the benzene skeleton in Formula (B) of the compound of Formula (I) is bound to the PYP or PYP-derived protein contained in the fusion protein, a binding site of, for example, the PYP has a space into which only the coumarin skeleton in formula (A) or the benzene skeleton in Formula (B) can get and the group X cannot get into the binding site of, for example, the PYP. As a result, the coumarin skeleton in Formula (A) or the benzene skeleton in Formula (B) is intramolecularly dissociated from the group X and therefore fluorescence derived from the group X can be emitted (see Scheme 2). In other words, the compound of Formula (I) or the salt thereof that is not bonded to the fusion protein has very low fluorescence intensity. That is, it does not emit fluorescence, which allows the background signals to be low in the labeling method of the present invention. As a result, after the target protein to be labeled (biomolecule) is labeled with the compound of Formula (I), even without washing, the target protein to be labeled can be detected accurately without any disturbance from background signals. Furthermore, the PYP or PYP-derived protein that is used in the labeling method of the present invention has a relatively low molecular weight, approximately 14 kDa, and therefore is unlikely to affect the target protein to be labeled, which is an advantage. Moreover, since the PYP or PYP-derived protein or the coumarin derivative or the coumaric acid derivative is a substance that does not exist in an animal cell, it is unlikely to affect the label, which is an advantage.

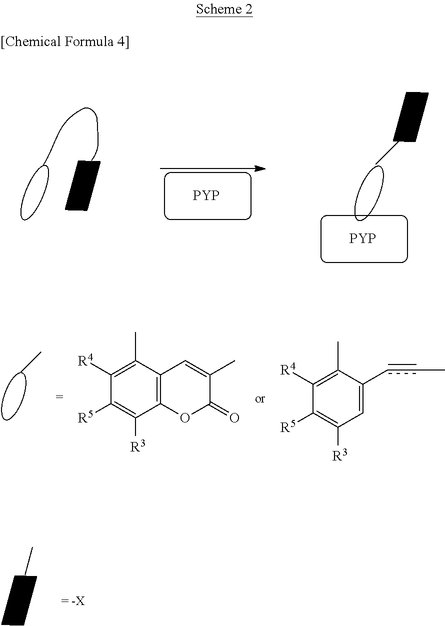

Examples of the amino acid sequence of the PYP include amino acid sequences represented by SEQ ID NOS: 1 to 17 of the sequence listing. Examples of the polynucleotide for coding the PYP include base sequences represented by SEQ ID NOS: 18 to 29 of the sequence listing.

In order to obtain a plasmid or vector that can express a fusion protein, for example, a polynucleotide that codes a target protein to be labeled or a polynucleotide that codes a PYP or PYP-derived protein can be used to prepare such a plasmid or vector according to an ordinary method. In the present invention, the vector is a polynucleotide, preferably a polynucleotide for transfecting a cell with a polynucleotide for coding the fusion protein, and embraces a plasmid. The vector may be a plasmid vector or a viral vector. A person skilled in the art suitably can select the sequence that allows the fusion protein to be expressed, according to the type of the cell to be transfected. The plasmid and the vector may contain a sequence (for example, an expression-inducing promoter or a regulatory sequence) that adjusts expression of the fusion protein.

The fusion protein can be expressed in a cell by using a polynucleotide for coding the fusion protein or a plasmid or vector that can express the fusion protein, according to an ordinary method.

An expressed fusion protein can be isolated according to an ordinary method.

As described above, in the compound of Formula (I) that is used in the fluorescently labeling method of the present invention, Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including the coumarin skeleton or a group represented by Formula (B) including the benzene skeleton,
wherein in Formula (A) and Formula (B),
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen,
R⁵ is a hydroxy group, an amino group, an amino group that has been mono- or di-substituted with lower alkyl, or a lower alkoxy group, and

[Chemical Formula 5]

[symbol]

denotes a double bond or a triple bond.

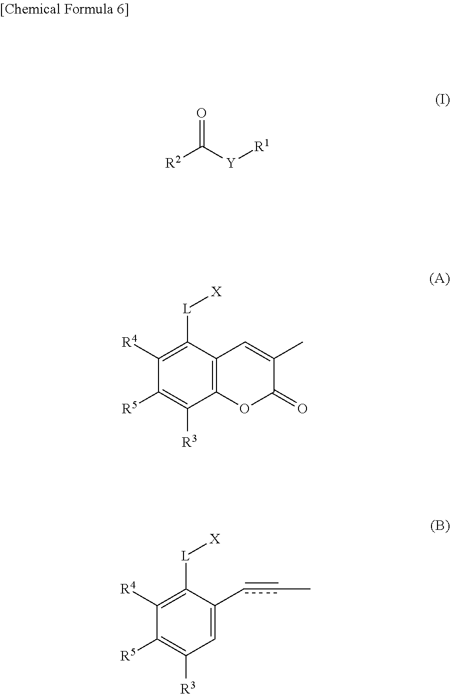

In the present invention, the fluorescent group denotes a group having properties in which when it is irradiated with, for example, visible light, ultraviolet rays, or X-rays, it absorbs energy thereof thereby electrons are excited, and it releases extra energy as an electromagnetic wave when it returns to the ground state. Specifically, examples of the fluorescent group include groups derived from, for example, fluorescein, a xanthene dye, a cyanine dye, acridine, isoindole, a dansyl dye, aminophthalic hydrazide, aminophthalimide, aminonaphthalimide, aminobenzofuran, aminoquinoline, or dicyanohydroquinone, or a luminous rare earth complex. Preferably, the fluorescent group is a group derived from fluorescein or a xanthene dye.

In the present invention, the linker L for X is a group for binding the group X and the coumarin skeleton of the group of Formula (A) or a group for binding the group X and the benzene skeleton of the group of Formula (B). Preferably, the linker L is flexible and long to some extent so that the coumarin skeleton of Formula (A) or the benzene skeleton of Formula (B) is intramolecularly associated with the group X. In order to be flexible, the linker L is preferably one having a chain structure. Furthermore, the length of the linker L is, for example, 8 Å to 25 Å, preferably 10 Å to 20 Å, and more preferably 12 Å to 18 Å. Specifically, examples of L include —(CH₂)ₙ₁O(CH₂)ₙ₂—, —(CH₂)ₙ₁CONR¹¹(CH₂)ₙ₂—, —(CH₂)ₙ₁NR¹¹CO(CH₂)ₙ₂—, —(CH₂)ₙ₁NR¹¹CONR¹²(CH₂)ₙ₂—, —(CH₂)ₙ₁NR¹¹CSNR¹²(CH₂)ₙ₂—, —(CH₂)ₙ₁CONR¹¹(CH₂)ₙ₃CONR¹²(CH₂)ₙ₂—, —(CH₂)ₙ₁NR¹¹CO(CH₂)ₙ₃NR¹²CO(CH₂)ₙ₂—, —(CH₂)ₙ₁—, —(CH₂)ₙ₁S(CH₂)ₙ₂—, —(CH₂)ₙ₁NR¹¹(CH₂)ₙ₂—, —(CH₂)ₙ₁CO₂(CH₂)ₙ₂—, and —(CH₂)ₙ₁O(CH₂)ₙ₂O(CH₂)ₙ₃O(CH₂)ₙ₄— as well as

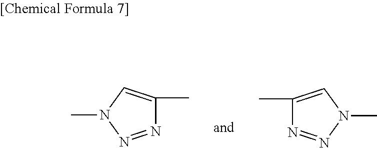

and those obtained by combining two or more of them. In the above-mentioned formulae, n1, n2, and n3 are each independently an integer of 0 or 1 to 5, and R¹¹ and R¹² each are a hydrogen atom or a lower alkyl group. The structure of the linker for X is not limited as long as the linker is not cleaved under conditions in the method for fluorescently labeling a protein of the present invention. The aforementioned linker L is preferably a group represented by Formula (a), Formula (b), Formula (c), or Formula (d) below.

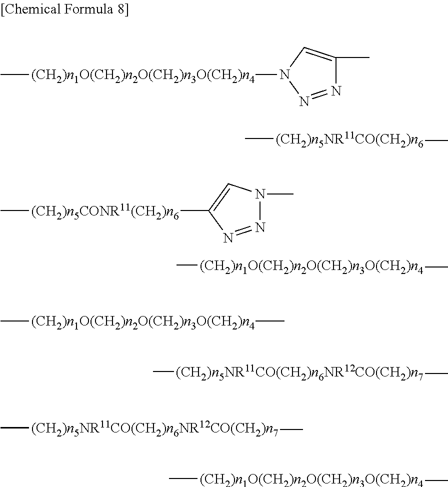

In Formula (a) and Formula (b), n1, n2, n3, n4, n5, and n6 are each independently an integer of 0 or 1 to 5, and R¹¹ is a hydrogen atom or a lower alkyl group, and

in Formula (c) and Formula (d), n1, n2, n3, n4, n5, n6, and n7 are each independently an integer of 0 or 1 to 5, and R¹¹ and R¹² are each independently a hydrogen atom or a lower alkyl group.

In the compound of Formula (I), R¹, R², a group of Formula (A), and a group of Formula (B) each may have at least one chiral center. Therefore, the compound of Formula (I) may be present as a racemate or a pure stereoisomer. Furthermore, when Formula (B) includes a double bond, it may be present as a cis or trans isomer. In each case, the present invention includes both mixtures thereof and respective isomers thereof.

In the present invention, the term “lower” denotes 1 to 6 carbon atoms and preferably 1 to 4 carbon atoms unless otherwise indicated.

In the present invention, examples of the “halogen atom” or “halogen” include a fluorine atom, a chlorine atom, a bromine atom, and an iodine atom.

In the present invention, examples of the “lower alkyl group” or “lower alkyl” include those having 1 to 6 carbon atoms. Specific examples of the lower alkyl group include linear or branched alkyl groups such as methyl, ethyl, n-propyl, i-propyl, n-butyl, i-butyl, sec-butyl, t-butyl, n-pentyl, i-pentyl, sec-pentyl, t-pentyl, 2-methylbutyl, n-hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, and 1-ethyl-1-methylpropyl, and preferably those having 1 to 3 carbon atoms.

In the present invention, examples of the “lower alkoxy group” or “lower alkoxy” include alkyloxy groups having 1 to 6 carbon atoms. Specific examples of the lower alkoxy groups include linear or branched alkyloxy groups such as methoxy, ethoxy, n-propyloxy, i-propyloxy, n-butyloxy, i-butyloxy, sec-butyloxy, t-butyloxy, n-pentyloxy, i-pentyloxy, sec-pentyloxy, t-pentyloxy, 2-methylbutoxy, n-hexyloxy, i-hexyloxy, t-hexyloxy, sec-hexyloxy, 2-methylpentyloxy, 3-methylpentyloxy, 1-ethylbutyloxy, 2-ethylbutyloxy, 1,1-dimethylbutyloxy, 2,2-dimethylbutyloxy, 3,3-dimethylbutyloxy, and 1-ethyl-1-methylpropyloxy, and preferably alkyloxy groups having 1 to 3 carbon atoms.

In the present invention, examples of the “aryl group” include those having 6 to 10 carbon atoms. Specific examples of the aryl group include a phenyl group and a naphthyl group and preferably a phenyl group.

In the present invention, examples of the “lower aralkyl group” include aryl-substituted lower alkyl groups. Specific examples of the lower aralkyl group include benzyl(phenylmethyl), phenethyl(phenylethyl), 3-phenylpropyl, 1-naphthylmethyl, and 2-(1-naphthyl)ethyl, and preferably a benzyl group.

In the present invention, examples of the “lower alkyl group substituted with halogen” include fluoromethyl, difluoromethyl, trifluoromethyl, chloromethyl, dichloromethyl, trichloromethyl, bromomethyl, dibromomethyl, tribromomethyl, 1-fluoroethyl, 1-chloroethyl, 1-bromoethyl, 2-fluoroethyl, 2-chloroethyl, 2-bromoethyl, 1,2-difluoroethyl, and tolufluoromethyl.

In the present invention, examples of the “amino group that has been mono- or di-substituted with lower alkyl” include methylamino, ethylamino, n-propylamino, i-propylamino, n-butylamino, dimethylamino, and diethylamino.

In the present invention, examples of the “aryl group substituted with at least one selected from the group consisting of hydroxy, halogen, and lower alkoxy” include 4-hydroxyphenyl, 4-chlorophenyl, 4-bromophenyl, 4-fluorophenyl, 2,6-dichlorophenyl, 4-methoxyphenyl, 3,4-dimethoxyphenyl, and 2-hydroxynaphthyl.

In the present invention, examples of the “lower aralkyl group substituted with at least one selected from the group consisting of hydroxy, halogen, and lower alkoxy” include 4-hydroxybenzyl, 2-chlorobenzyl, 2-bromobenzyl, and 4-methoxybenzyl.

The salt of the compound of Formula (I) may be a salt with acid or base. Examples of such a salt include salts of alkali metal such as sodium and potassium, salts of alkaline earth metal such as calcium and magnesium, salts with inorganic bases such as ammonium salts, salts with organic amine such as triethylamine, pyridine, picoline, ethanolamine, triethanolamine, dicyclohexylamine, and N,N′-dibenzylethyleneamine, salts with inorganic acid such as hydrochloric acid, hydrobromic acid, sulfuric acid, and phosphoric acid, salts with organic carboxylic acid such as formic acid, acetic acid, trifluoroacetic acid, maleic acid, and tartaric acid, addition salts with sulfonic acid such as methanesulfonic acid, benzenesulfonic acid, and p-toluenesulfonic acid, salts with bases such as basic or acidic amino acid, such as arginine, aspartic acid, and glutamic acid, as well as acid addition salts.

The compound of Formula (I) may be in the form of a solvate and this also is included in the range of the present invention. Examples of the solvate include preferably a hydrate and an ethanolate.

The compound of Formula (I) that is used in the fluorescently labeling method of the present invention may be produced in house by reference to known documents of conventional techniques or may be obtained commercially.

Obtaining a fusion protein of a target protein to be labeled and a PYP or PYP-derived protein, which is used in the fluorescently labeling method of the present invention, may include, for example, obtaining a polynucleotide for coding the fusion protein, obtaining a plasmid or vector that can express the fusion protein, expressing the fusion protein in a cell, or isolating the fusion protein thus expressed. With, for example, a polynucleotide for coding a target protein to be labeled or a polynucleotide for coding a PYP or PYP-derived protein being used for the polynucleotide for coding the fusion protein, a plasmid or vector that can express a fusion protein can be prepared according to the ordinary method.

In the method for fluorescently labeling a protein of the present invention, a step of reacting the fusion protein with the compound of Formula (I) may be carried out in a cell or in vivo where the fusion protein is expressed or may be carried out in vitro using an isolated fusion protein. When carried out in vitro, labeling may be carried out, for example, in a buffer (pH 6.8 to 8.5) at a temperature of 20 to 37° C.

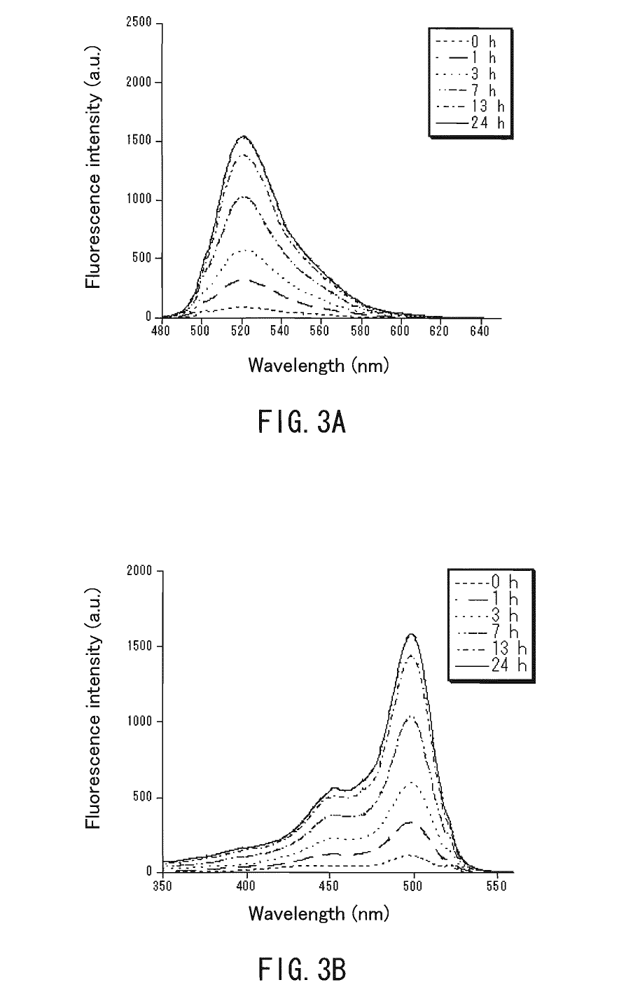

In the method for fluorescently labeling a protein of the present invention, the amino acid sequence of the PYP or PYP-derived protein in the fusion protein is preferably one selected from the group consisting of:
1) amino acid sequences represented by SEQ ID NOS: 1 to 17 of the sequence listing,
2) amino acid sequences represented by SEQ ID NOS: 1 to 17 of the sequence listing in which the 1st to 24th, 25th, 26th, or 27th amino acids have been deleted,
3) amino acid sequences in which one to several amino acids of the amino acid sequences of either 1) or 2) described above have been deleted, substituted, or added, wherein when the fusion protein binds to the coumarin skeleton in Formula (A) or the benzene skeleton in Formula (B), the coumarin skeleton in Formula (A) or the benzene skeleton in Formula (B) is intramolecularly dissociated from the group X in the compound represented by Formula (I), and thereby fluorescence derived from the group X can be emitted, and
4) amino acid sequences having a homology of at least 70% with the amino acid sequences of either 1) or 2) described above, wherein when the fusion protein binds to the coumarin skeleton in Formula (A) or the benzene skeleton in Formula (B), the coumarin skeleton in Formula (A) or the benzene skeleton in Formula (B) is intramolecularly dissociated from the group X in the compound represented by Formula (I), and thereby fluorescence derived from the group X can be emitted. In this case, the amino acid sequences represented by SEQ ID NOS: 1 to 17 of the sequence listing are those of PYPs that are present in 15 types of purple bacteria. Furthermore, according to the document, Kumauchi et al. Photochemistry and Photobiology, 2008, 84:956-969, the PYPs of the purple bacteria are known to exhibit ligand-binding affinity even when the 24th to 27th amino acid sequences at the N terminus are deleted. A person skilled in the art can easily determine the sites that can be deleted at the N terminus with reference to the multiple alignment shown in FIG. 2(a) in the document described above.

The fusion protein of the target protein to be labeled and the PYP or PYP-derived protein can be obtained by a method including at least one or all of the steps of: obtaining a polynucleotide for coding the fusion protein, obtaining a plasmid or vector that can express the fusion protein, expressing the fusion protein in a cell, or isolating the fusion protein thus expressed. That is, obtaining the fusion protein includes not only isolating the protein but also expressing the fusion protein in a cell or in vivo.

The present invention also is a composition to be used for a method of the present invention containing a compound represented by Formula (I) or a salt thereof.

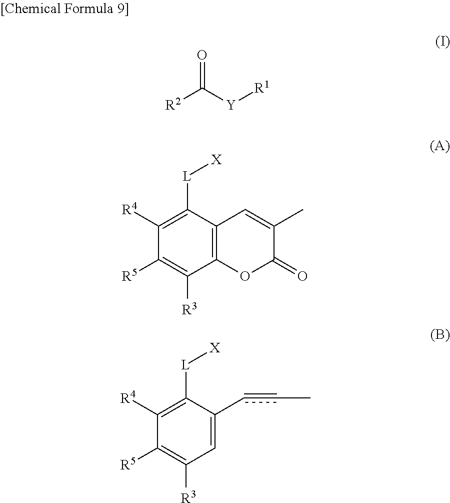

In Formula (I),
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including a coumarin skeleton or a group represented by Formula (B) including a benzene skeleton,
wherein in Formula (A) and Formula (B),
X is a fluorescent group,
L is a linker for X,
R³ and R⁴ are each independently a hydrogen atom, a halogen atom, a lower alkyl group, or a lower alkyl group substituted with halogen,
R⁵ is a hydroxy group, an amino group, an amino group that has been mono- or di-substituted with lower alkyl, or a lower alkoxy group, and

[Chemical Formula 10]

[symbol]

denotes a double bond or a triple bond.

The present invention also is a plasmid or vector to be used for a method of the present invention,

wherein the plasmid or vector is one for cloning a polynucleotide for coding a fusion protein of a target protein to be labeled and a PYP or PYP-derived protein or one for expressing the fusion protein, and

the plasmid or vector includes a base sequence selected from the group consisting of:
1) base sequences represented by SEQ ID NOS: 18 to 29 of the sequence listing,
2) base sequences represented by SEQ ID NOS: 18 to 29 of the sequence listing in which base sequences have been deleted corresponding to deletion of the 1st to 24th, 25th, 26th, or 27th at the N terminus of the amino acid sequences that are coded with the aforementioned base sequences,
3) base sequences in which one to several bases of the base sequences of either 1) or 2) described above have been deleted, substituted, or added, wherein the base sequences code amino acid sequences in which when the fusion protein binds to the coumarin skeleton in Formula (A) or the benzene skeleton in Formula (B), the coumarin skeleton in Formula (A) or the benzene skeleton in Formula (B) is intramolecularly dissociated from the group X in the compound represented by Formula (I), and thereby fluorescence derived from the group X can be emitted, and
4) base sequences that have a homology of at least 70% with the base sequences of either 1) or 2) described above, wherein the base sequences code amino acid sequences in which when the fusion protein binds to the coumarin skeleton in Formula (A) below or the benzene skeleton in Formula (B) below, the coumarin skeleton in Formula (A) or the benzene skeleton in Formula (B) is intramolecularly dissociated from the group X in the compound represented by Formula (I), and thereby fluorescence derived from the group X can be emitted. The base sequences represented by SEQ ID NOS: 18 to 29 of the sequence listing are those for coding amino acid sequences of PYPs represented by SEQ ID NOS: 1 to 12 of the sequence listing, respectively. Deletion of 5′-side base sequences in the above-mentioned base sequences 2) is the same as described above.

Furthermore, the present invention is a kit for a method for fluorescently labeling a protein. The kit includes a plasmid or vector to be used for a method of the present invention and a composition to be used for a method of the present invention containing a compound represented by Formula (I) or a salt thereof.

Preferably, the kit for a method for fluorescently labeling a protein of the present invention further includes a host cell to be transfected with the plasmid or vector and an instruction manual for the kit.

Preferably, in the method, composition, and kit of the present invention,
Y is a sulfur atom,
R¹ is an aryl group or an aryl group substituted with at least one selected from the group consisting of hydroxy, halogen, and lower alkoxy, and
R² is a group represented by Formula (A) or Formula (B),
wherein in Formula (A) and Formula (B),
X is a group represented by Formula (i),
L is a group represented by Formula (a), Formula (b), Formula (c), or Formula (d),
R³ and R⁴ each are a hydrogen atom,
R⁵ is hydroxy, and

[Chemical formula 11]

[symbol]

denotes a double bond,
wherein in Formula (a) and Formula (b),
n1, n2, n3, n4, n5, and n6 are each independently an integer of 0 or 1 to 5, and
R¹¹ is a hydrogen atom or a lower alkyl group, and

in Formula (c) and Formula (d),
n1, n2, n3, n4, n5, n6, and n7 are each independently an integer of 0 or 1 to 5, and
R¹¹ and R¹² are each independently a compound of Formula (I), which is a hydrogen atom or a lower alkyl group, or a salt thereof.

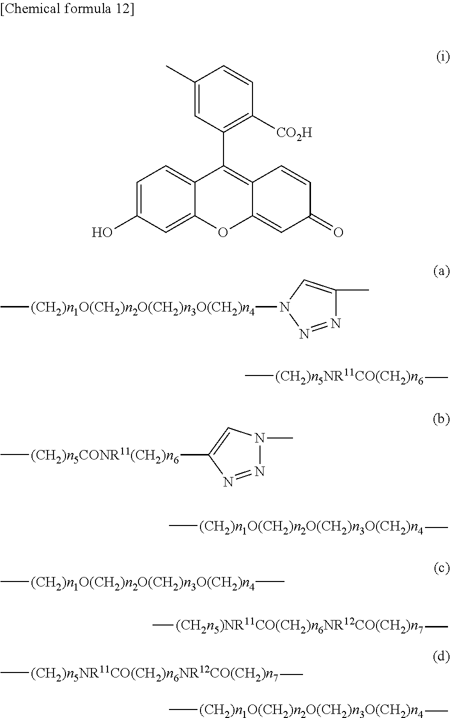

Moreover, the present invention is a compound represented by General Formula (I) or a salt thereof.

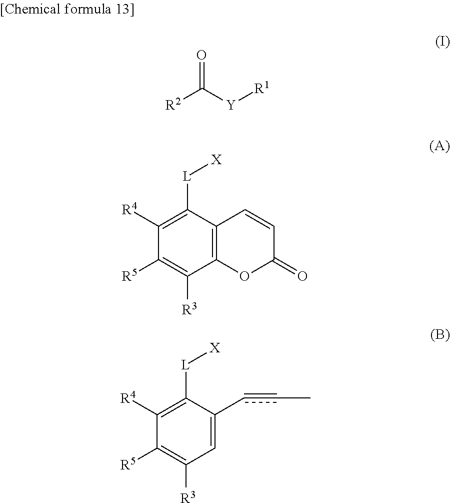

In Formula (I) above,
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including a coumarin skeleton or a group represented by Formula (B) including a benzene skeleton,
wherein in Formula (A) and Formula (B),
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen,
R⁵ is a hydroxy group, an amino group, an amino group that has been mono- or di-substituted with lower alkyl, or a lower alkoxy group, and

[Chemical formula 14]

[symbol]

denotes a double bond or a triple bond.

Preferably, with respect to the compound of Formula (I), in Formula (I),
Y is a sulfur atom,
R¹ is an aryl group or an aryl group substituted with at least one selected from the group consisting of hydroxy, halogen, and lower alkoxy, and
R² is a group represented by Formula (A) or Formula (B),
wherein in Formula (A) and Formula (B),
X is a group represented by Formula (i),
L is a group represented by Formula (a), Formula (b), Formula (c), or Formula (d),
R³ and R⁴ each are a hydrogen atom,
R⁵ is hydroxy, and

[Chemical formula 15]

[symbol]

denotes a double bond,
wherein in Formula (a) and Formula (b),
n1, n2, n3, n4, n5, and n6 are each independently an integer of 0 or 1 to 5, and
R¹¹ is a hydrogen atom or a lower alkyl group, and

in Formula (c) and Formula (d),
n1, n2, n3, n4, n5, n6, and n7 are each independently an integer of 0 or 1 to 5, and
R¹¹ and R¹² are each independently a hydrogen atom or a lower alkyl group.

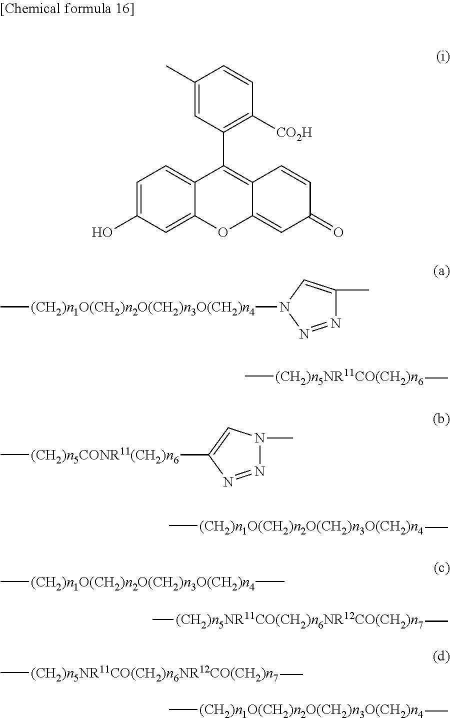

Furthermore, the compound of Formula (I) is more preferably a compound FCTP represented by the following formula. These preferred compounds of Formula (I) are also used preferably in, for example, a method for fluorescently labeling a protein and a composition of the present invention.

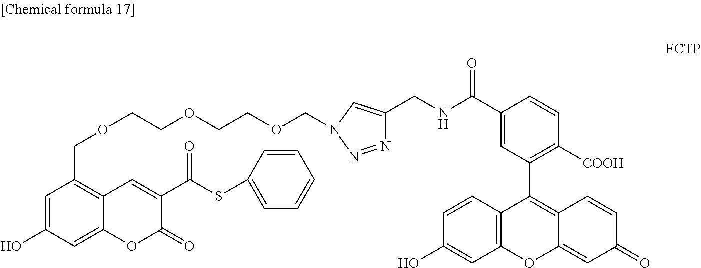

The compound of Formula (I) can be produced, for example, according to Scheme 3 below. Scheme 3 below shows a production example wherein in Formula (I), L is a specific group. The starting material may be produced in house by reference to known documents of conventional techniques or may be purchased.

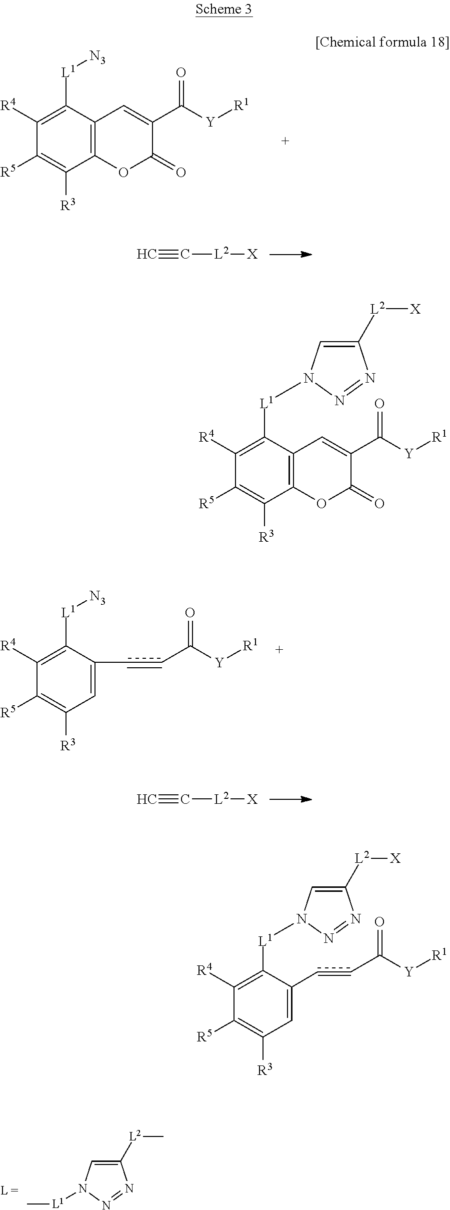

In the above-mentioned formula,
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy,
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen,
R⁵ is a hydroxy group, an amino group, an amino group that has been mono- or di-substituted with lower alkyl, or a lower alkoxy group, and

[Chemical formula 19]

[symbol]

denotes a double bond or a triple bond.

The present invention further can provide a bioimaging method that includes labeling a target protein by a method for fluorescently labeling a protein of the present invention. Preferably, the bioimaging method of the present invention includes imaging of a cell or tissue that expresses the target protein. The cell or tissue is not particularly limited but is preferably an animal cell or tissue, more preferably a mammalian cell or tissue, further preferably a primate cell or tissue, and still further preferably a human cell or tissue.

### EXAMPLES

The following abbreviations are used in the descriptions in this specification.
DMSO: Dimethylsulfoxide
HOBt: 1-hydroxybenzotriazole
DMF: Dimethylformamide
WSCD•HCl: Water-soluble carbodiimide:
1-ethyl-3(3-dimethylaminopropyl)carbodiimide hydrochloride
MOMCl: Methoxymethylchloride
DIEA: N,N-diisopropylethylamine
NBS: N-bromosuccinimide
AIBN: Azobisbutyronitrile
TFA: Trifluoroacetic acid
TCEP: Tris[2-carboxyethyl]phosphine
SDS-PAGE: Sodium dodecyl sulfate polyacrylamide gel electrophoresis
CBB: Coomassie brilliant blue
DTT: Dithiothreitol
MESNa: 2-mercaptoethanesulfonic acid sodium salt

[Compounds and Instruments]

Compounds were of the best grade available, purchased from Tokyo Chemical Industry Co., Ltd., Wako Pure Chemical Industries, Ltd., and Sigma-Aldrich Japan, K.K., and used without further purification.

NMR spectra were recorded on a JEOL JNM-AL400 instrument (JEOL Ltd.) at 400 MHz for ¹H and at 100.4 MHz for ¹³NMR using tetramethylsilane as an internal standard. Mass spectra (EI) were taken on a Shimazu GCMS-QP2000 (Shimadzu Corporation) operating in the electron impact mode (70 eV) equipped with CBP1-M25-025 column. High-resolution mass spectra (HRMS) were obtained on a JEOL JMS-DX303 (JEOL Ltd.). ESI-TOF MS was taken on a Waters LCT-Premier XE (Nihon Waters K.K.). Fluorescence spectra were measured using a Hitachi F4500 spectrometer (Hitachi, Ltd.). Slit width was 2.5 nm for both excitation and emission. The photomultiplier voltage was 700 V. Samples were dissolved in DMSO (biochemical grade, Wako Pure Chemical Industries, Ltd.) before fluorescence measurement. Silica gel column chromatography was performed using BW-300 (Fuji Silysia Chemical Ltd.). Fluorescence imaging was visualized using AE-6935B VISIRAYS-B.

### Example 1

Production of Compound FCTP

The following compound FCTP was produced according to Scheme 4 below.

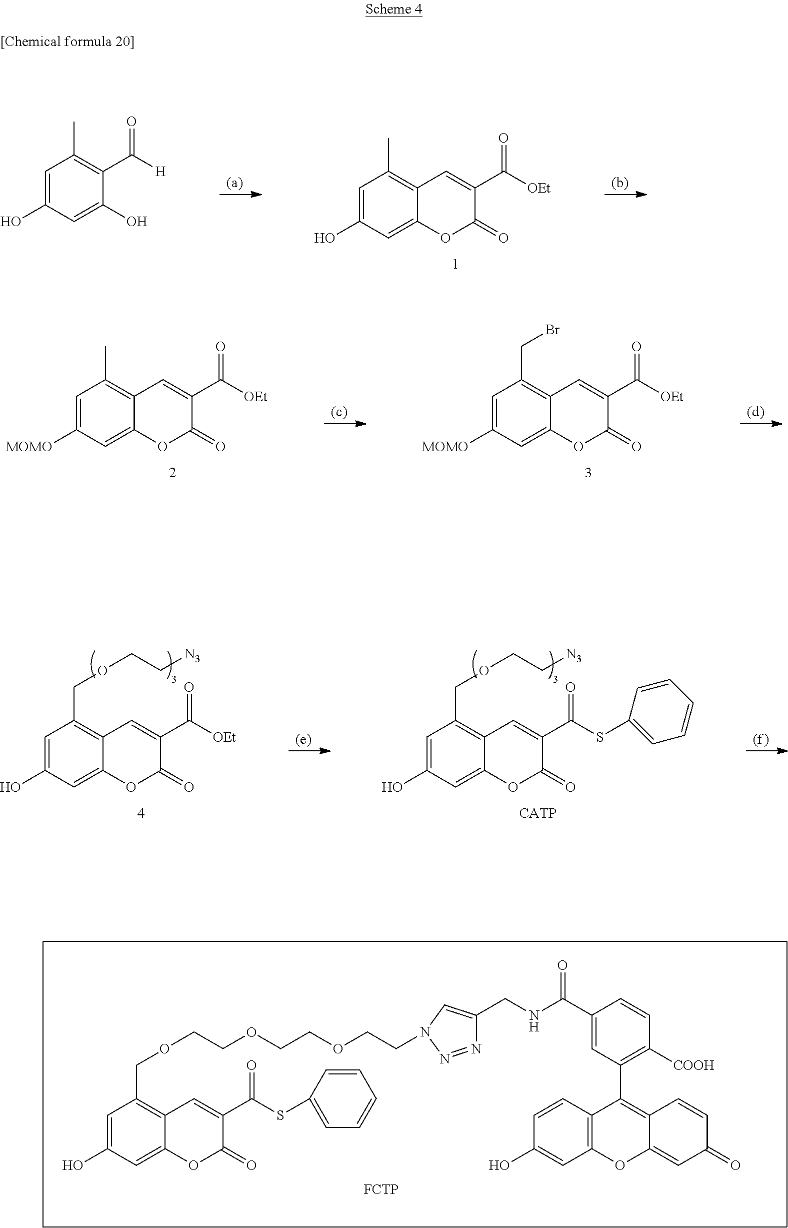

In Scheme 4: (a) diethyl malonate, piperidine/EtOH, (b) MOMCl, DIEA/DMF, (c)NBS, AIBN/CCl₄, (d) 1. 2-[2-(2-azidoethoxy)ethoxy]ethanol, NaH/DMF; 2.2N aqueous NaOH solution; 3. TFA, (e) thiophenol, WSCD.HCl, HOBt.H₂O/DMF, (f) 6-carboxyfluorescein-propargylamide, CuSO₄, sodium ascorbate/DMF/H₂O (4/1).

### Production of 7-hydroxy-5-methylcoumarin-3-carboxylic acid ethyl ester (1)

2,4-dihydroxy-6-methylbenzaldehyde (2.0 g), diethyl malonate (2.2 ml, 14.4 mmol), and piperidine (0.35 ml) were dissolved in ethanol (20 ml), which then was stirred at 100° C. for 23 hours. After the mixture was cooled, the crystals obtained thereby were isolated by filtration and washed with cold ethanol (−20° C.) to yield compound (1) (2.02 g, yield 62%).

¹H-NMR (400 MHz, CDCl₃): δ 1.41 (t, J=7.2 Hz, 31⁻1), 2.54 (s, 3H), 4.41 (q, J=7.2 Hz, 2H), 6.71 (d, J=7.2 Hz, 1H) 6.76 (d, 1H), 8.71 (s, 1H);

¹³C-NMR (100 MHz, CDCl₃): δ 14.1, 17.9, 60.8, 99.9, 109.3, 110.9, 115.2, 140.6, 146.0, 156.4, 157.9, 163.3, 164.2;

HRMS(EI⁺): Calculated 248.06, Found 248.06.

### Production of 7-(methoxymethoxy)-5-methylcoumarin-3-carboxylic acid ethyl ester (2)

7-hydroxy-5-methylcoumarin-3-carboxylic acid ethyl ester (1) (0.20 g, 0.7 mmol), diisopropylethylamine (0.40 ml, 18 mmol), and chloromethylmethylether (0.015 ml, 1.8 mmol) were dissolved in DMF, which then was stirred at 0° C. for two hours. After the solvent was removed from the mixture, the residue thus obtained was dissolved in a 10% aqueous citric acid solution (30 mL) and extracted with ethyl acetate. The organic phase thus obtained was dried over Na₂SO₄ and the solvent was evaporated to dryness under reduced pressure to yield compound (2) (0.186 mg, yield 79%).

¹H-NMR (400 MHz, CDCl₃); δ 1.41 (t, J=7.2 Hz, 3H) 2.55 (s, 1H) 3.49 (s, 3H) 4.42 (q, J=7.2 Hz, 2H) 5.23 (s, 2H) 6.82 (d, J=6.0 Hz, 1H) 6.85 (d, J=6.0 Hz, 1H) 8.69 (s, 1H);

¹³C-NMR (100 MHz, CDCl₃): δ 14.3, 18.5, 56.5, 61.7, 94.2, 101.2, 111.7, 113.8, 115.3, 139.8, 145.9, 157.1, 157.9, 162.1, 163.8;

HRMS(EI⁺): Calculated 292.09, Found 292.09.

### Production of 5-bromomethyl-7-(methoxymethoxy)coumarin-3-carboxylic acid ethyl ester (3)

7-(methoxymethoxy)-5-methylcoumarin-3-carboxylic acid ethyl ester (2) (0.5 g, 1.3 mmol), NBS (0.365 g, 2.1 mmol), and AIBN (0.04 g, 0.24 mmol) were dissolved in CCl₄ (15 ml), which then was stirred at 95° C. for seven hours. After the solvent was removed from the mixture, the residue thus obtained was eluted with dichloromethane/ethyl acetate (97/3) and purified using silica gel column chromatography to yield compound (3) (0.327 g, yield 52%).

¹H-NMR (400 MHz, CDCl₃); δ 1.42 (t, J=7.2 Hz, 3H) 3.50 (s, 3H) 4.42 (q, J=7.2 Hz, 2H) 4.65 (s, 1H) 5.27 (s, 2H) 6.98 (d, J=6.0 Hz, 1H) 7.01 (d, J=6.0 Hz, 1H) 8.77 (s, 1H);

¹³C-NMR(100 MHz, CDCl₃); δ 14.3, 27.9, 56.7, 62.0, 94.5, 103.7, 110.7, 114.8, 115.7, 138.1, 144.5, 156.4, 157.9, 161.7, 163.3;

HRMS(EI⁺) Calculated 370.01, Found 370.00.

### Production of 5-((2-(2-(2-azidoethoxy)ethoxy)ethoxy)methyl)-7-hydroxycoumarin-3-carboxylic acid (4)

2-[2-(2-azidoethoxy)ethoxy]ethanol (0.233 g, 1.3 mmol) and sodium hydride (0.038 g, 1.6 mmol) were dissolved in DMF (5 ml), which then was stirred at room temperature for 15 minutes. 5-bromomethyl-7-(methoxymethoxy)coumarin-3-carboxylic acid ethyl ester (3) was added to the mixture, and the mixture thus obtained was stirred at room temperature for 1.5 hours. Then, an aqueous NaOH solution (2 N, 3 ml) was added to the mixture. After 20 minutes, the mixture was neutralized with a saturated solution of citric acid and with dichloromethane. After the solvent was removed, the residue thus obtained was dissolved in TFA (10 ml), which then was stirred at room temperature for one hour. After the solvent was removed, the residue obtained by reversed-phase HPLC in which it was eluted with H₂O/acetonitrile containing 0.1% formic acid was purified to yield compound (4) (21 mg, yield 8%).

¹H-NMR (400 MHz, CDCl₃): δ 3.21 (s, 2H) 3.53-3.61 (m, 10H) 6.61 (s, 1H) 6.82 (s, 1H) 8.86 (s, 1H);

MS(ESI⁺) Calculated 394.35, Found 394.11.

Production of Compound CATP

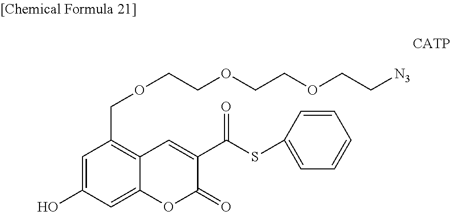

5-((2-(2-(2-azidoethoxy)ethoxy)ethoxy)methyl)-7-hydroxycoumarin-3-carboxylic acid (4) (15.5 mg, 0.039 mmol), WSCD.HCl (9.6 mg, 0.047 mmol), and HOBt.H₂O (7.3 mg, 0.047 mmol) were dissolved in DMF (2 ml), which then was stirred at room temperature for one hour. Thereafter, thiophenol (0.0039 ml, 0.039 mmol) was added to the mixture, which then was stirred for one hour. After the solvent was removed, the residue thus obtained was eluted with dichloromethane/methanol (98/2) and purified by silica gel column chromatography to yield compound CATP (17 mg, 89%).

¹H-NMR (400 MHz, CDCl₃): δ 3.37 (t, J=4.8 Hz, 2H) 3.60-3.75 (m, 10H) 4.73 (s, 2H) 6.71 (d, J=2.0 Hz, 1H) 7.01 (d, J=2.0 Hz, 1H) 7.46 (m, 5H) 8.68 (s, 1H);

¹³C-NMR (100 MHz, CDCl₃): δ 50.5, 69.6, 69.8, 70.1, 70.2, 70.6, 77.0, 70.6, 102.4, 109.6, 114.5, 117.6, 128.3, 129.1, 129.5, 134.9, 140.5, 158.1, 159.2, 163.4, 187.2;

HRMS (FAB⁺) Calculated 486.14, Found 486.13.

Production of Compound FCTP

Compound CATP (32 mg, 0.068 mmol), 6-carboxyfluorescein-propargylamide (28 mg, 0.068 mmol), CuSO₄ (8.5 mg, 0.034 mmol), and sodium ascorbate (13.3 mg, 0.068 mmol) were dissolved in DMF/H₂O (4/1), which then was stirred at room temperature for one hour. After the solvent was removed, the residue obtained by reversed-phase HPLC in which it was eluted with 0.1% formic acid-containing H₂O/acetonitrile was purified to yield Compound FCTP (10 mg, yield 16%).

¹H-NMR (400 MHz, DMSO-d₆): δ 3.35-4.35 (m, 8H) 3.70 (t, J=4.8, 2H) 4.37-4.42 (m, 4H) 4.69 (s, 2H) 6.53-6.69 (m, 7H) 6.80 (s, 1H) 7.46 (s, 5H) 7.71 (s, 1H) 7.86 (s, 1H) 8.05 (d, 1H) 8.18 (d, 1H) 8.66 (s, 1H);

¹³C-NMR (100 MHz, DMSO-d₆): δ 34.9, 49.2, 68.6, 69.2, 69.3, 69.4, 69.5, 69.6, 69.7, 83.4, 101.7, 102.2, 108.3, 108.4, 109.1, 112.7, 115.8, 115.9, 122.3, 122.4, 124.8, 128.3, 128.4, 129.1, 129.2, 129.3, 129.4, 129.5, 134.8, 140.3, 140.9, 144.2, 151.8, 152.7, 158.4, 159.6, 164.4, 168.0, 185.3;

HRMS (FAB⁺) Calculated 899.22, Found 899.23.

[Cloning of PYP]

With a plasmid (for coding DNA of a His tag fusion protein of a PYP) used as a template, DNA of the His-PYP was amplified by PCR (PrimeSTAR HS DNA Polymerase, TAKARA BIO INC.) using the primers indicated below. The plasmid was provided by Professor K. J. Hellingwerf of University of Amsterdam. The PYP is derived from Ectohiodospira halophila (Halorhodospira halophila). The PCR fragment (insert) was separated with a 2.0% agarose gel and then extracted using a kit (QIA quick Gel Extraction kit, QIAGEN). The vector for E. coli expression, pET-21b(+), and the insert were subjected to a restriction enzyme treatment with Hindl III and Nde I (both manufactured by TAKARA BIO INC.), and a T4-DNA ligase was used to integrate the insert into pET-21b(+). Thus, the pET-PYP was produced.

[Chemical Formula 22]

### Primers for PCR

Forward Primer (SEQ ID NO: 30):
CGGAGATATACATATGAGAGGATCGCATCAC
Reverse Primer (SEQ ID NO: 31):
GCTAATTAGCTTCTAGACGC

[PYP Expression and Purification]

The pET-PYP was transformed into E. coli (BL21 (DE3)) and then grown to an OD600 of 0.6 to 0.8 Å in Luria-Bertani (LB) medium at 37° C. Next, IPTG was added with a final concentration of 100 μM and this was grown overnight at 20° C. The E. coli thus grown was harvested and then was subjected to ultrasonic fragmentation. Thereafter, a soluble fraction was extracted. The soluble fraction PYP was purified with the Ni-NTA column (Ni-NTA agarose; QIAGEN) using the buffers described below according to the QIAGEN's protocol. Then the fraction was subjected to final purification by gel filtration column chromatography (Superdex™ 7510/300 GL, GE Healthcare) using Tris buffer (20 mM Tris-HCl, 150 mM NaCl, 1 mM TCEP, pH 8.0) as a mobile phase.

Conditions for Ni-NTA column include:

a binding solution: phosphoric acid buffer (50 mM sodium phosphate, 300 mM NaCl, 10 mM imidazole, pH 8.0),

a washing solution: phosphoric acid buffer (50 mM sodium phosphate, 300 mM NaCl, 20 mM imidazole, pH 8.0), and

an eluate: phosphoric acid buffer (50 mM sodium phosphate, 300 mM NaCl, 50 mM imidazole, pH 8.0).

[Treatment of PYP with Enterokinase]

A sequence to be cleaved by a protease, enterokinase, has been inserted between His tag and PYP. In order to remove the His tag, enterokinase (Recombinant Enterokinase; Novagen) was added to the purified PYP, which then was allowed to stand at room temperature for 18 hours. Thereafter, a sample was added to the Ni-NTA column (Ni-NTA agarose; QIAGEN) and the His tag site was adsorbed thereto. Thus, cleaved PYP was purified. The sample from which the His tag site had been removed was added to a bent uridine column (HiTrap Benzamidine FF, GE Healthcare) and thereby protease was removed. The purified sample was subjected to ultrafiltration (VIVASPIN 500, 5000 MWCO PES, Sartorius Stedim) to substitute Tris buffer (20 mM Tris-HCl, 150 mM NaCl, 1 mM TCEP, pH 8.0).

[Reaction Between PYP and FCTP]

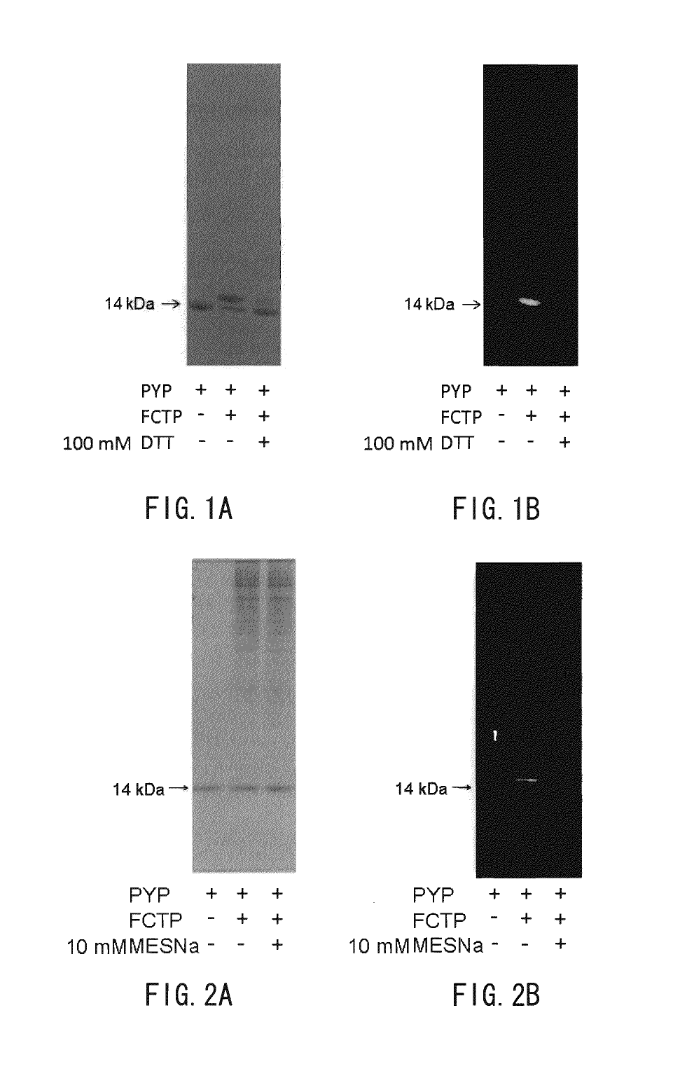

In Tris buffer (20 mM Tris-HCl, 150 mM NaCl, 1 mM TCEP (pH 8.0)), 5 μM of PYP was reacted with 12.5 μM of compound FCTP at 37° C. for 24 hours. After completion of the reaction, the result was analyzed by SDS-PAGE (20% acrylamide separation gel). The resultant CBB-stained image is shown in FIG. 1(a) and the resultant fluorescent image is shown in FIG. 1(b). With reference to FIG. 1(a), in the CBB-stained image, a slowly migrating band was observed with the addition of compound FCTP. With reference to FIG. 1(b), similarly in the fluorescent image, fluorescence was observed. Thus, it was confirmed that compound FCTP was able to label the PYP. Furthermore, since an addition of DTT (100 mM) reduced the slowly migrating band and also made the fluorescence intensity weaker, it was confirmed that the thioester bond between the PYP and compound CATP was hydrolyzed by a large excess of DTT.

[Reaction of Compound FCTP With PYP in Cell Lysate]

In a HEK293T cell lysate dissolved in Tris buffer (20 mM Tris-HCl, 150 mM NaCl, 1 mM TCEP (pH 8.0)), 5 μM of PYP was reacted with 50 μM of compound FCTP at 37° C. for 30 minutes in the presence or absence of 10 mM MESNa. After completion of the reaction, it was analyzed by SDS-PAGE (20% acrylamide separation gel). The resultant CBB-stained image is shown in FIG. 2(a) and the resultant fluorescent image is shown in FIG. 2(b). As shown in FIGS. 2(a) and 2(b), with an addition of compound FCTP, fluorescence was observed in the vicinity of the PYP band confirmed by the CBB-stained image. Furthermore, another fluorescent band was observed in the high molecular weight range. On the other hand, the fluorescent band on the high molecular weight side disappeared upon addition of MESNa (10 mM), which was a type of thiol compound. It was confirmed that an addition of a certain amount of free thiol allowed labeling to be carried out specifically in the cell lysate.

[Fluorescent Properties of FCTP]

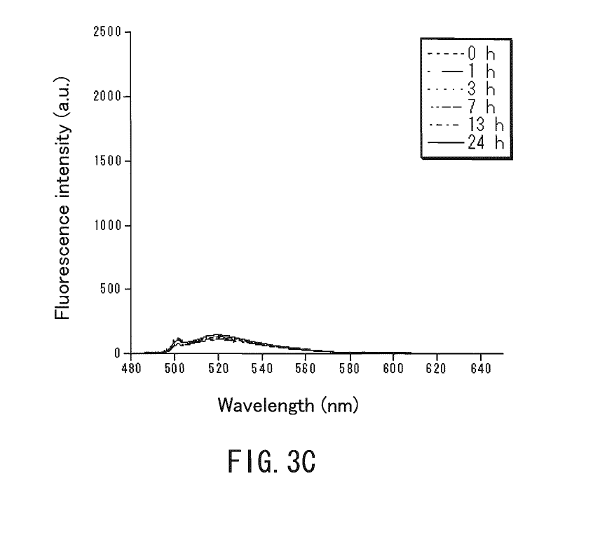

In order to examine the fluorescent properties of compound FCTP bound to the PYP, Tris buffer (20 mM Tris-HCl, 150 mM NaCl, 1 mM TCEP (pH 8.0)), the fluorescence and excitation spectra were measured at 37° C. FIG. 3(a) shows time-dependent fluorescence spectra of 8 μM of compound FCTP in the presence of 5 μM of PYP, FIG. 3(b) shows time-dependent excitation spectra of 8 μM of compound FCTP in the presence of 5 μM of PYP, and FIG. 3(c) shows fluorescence spectra (excitation at 500 nm) of 8 μM of compound FCTP.

As shown in FIG. 3(c), in the case of compound FCTP alone, the fluorescence intensity was very weak and no change in fluorescence spectra was observed with the passage of time On the other hand, as shown in FIGS. 3(a) and 3(b), the fluorescence intensity of compound FCTP increased in the presence of the PYP with the passage of time and changes in both fluorescence and excitation spectra were observed. From these results, it was confirmed that compound FCTP had a weaker fluorescence intensity before binding to the PYP and the fluorescence intensity increased considerably upon binding.

[Labeling Reaction with FCTP in Cultured Cell]

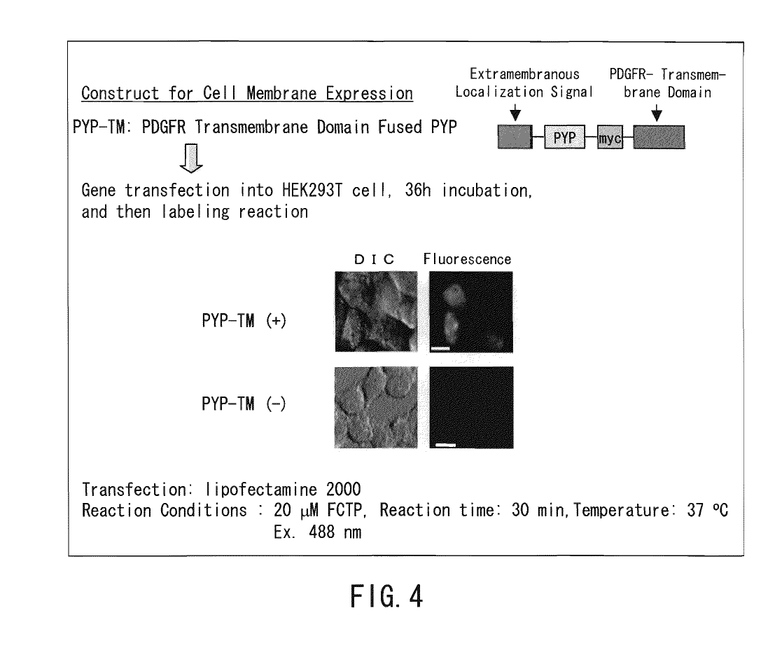

In order to express a PYP on the cell surface, a plasmid was prepared in which a gene PYP-TM obtained by fusing a PYP with the transmembrane domain of a platelet-derived growth factor receptor (PDGFR) was introduced into the multicloning site of pcDNA3.1, which is a mammalian expression vector. HEK293T cells were transfected with this plasmid by using lipofectamine 2000. After transfection with the plasmid, the HEK293T cells were allowed to stand at 37° C. for 36 hours and then washed with a phosphoric acid buffer (PBS). A culture medium containing FCTP (20 μM) was added thereto, which then was allowed to stand for 30 minutes. Thereafter, the culture media was replaced by a culture medium containing 10% fetal bovine serum (FBS) and the cells then were observed with a fluorescence microscope. With the fluorescence microscope having an excitation wavelength of 488 nm, the cells were observed using a fluorescent filter of 510 to 550 nm. The microscopic images obtained thereby are shown in FIG. 4.

[Labeling Reaction With CATP in Cultured Cell]

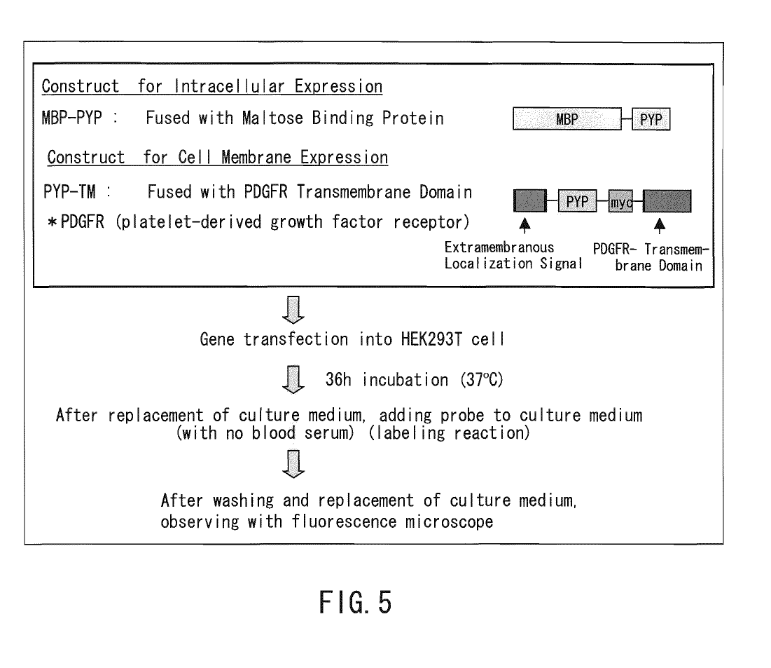

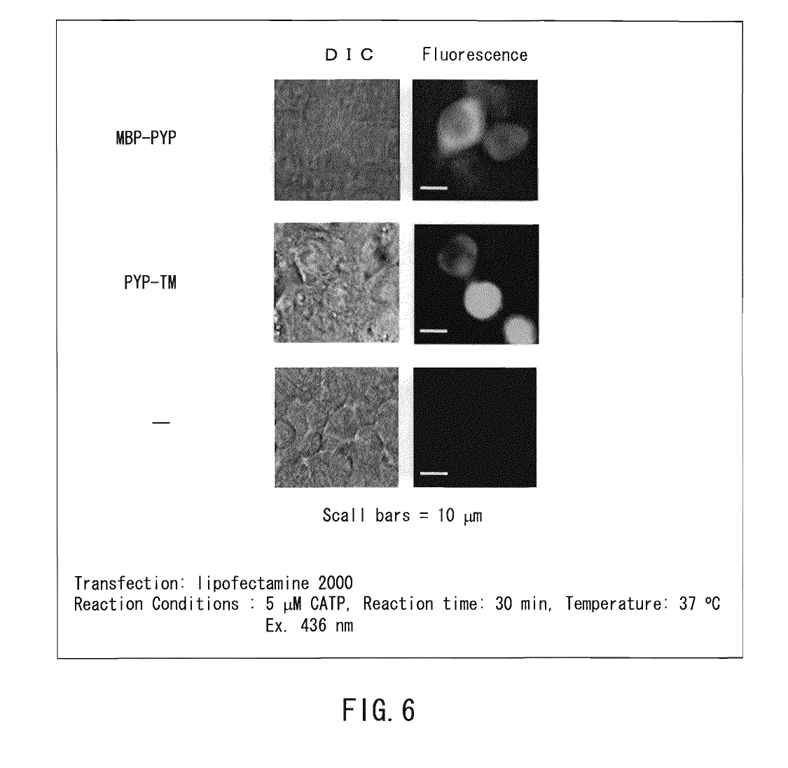

A plasmid was prepared in which a gene MBP-PYP obtained by fusing a maltose binding protein (MBP) with a PYP was introduced into the multicloning site of pcDNA3.1, which was a mammalian expression vector. Furthermore, in order to express a PYP on the cell surface, a plasmid was prepared in which a gene PYP-TM obtained by fusing a PYP with the transmembrane domain of a platelet-derived growth factor receptor (PDGFR) was introduced into the multicloning site of pcDNA3.1, which was a mammalian expression vector. HEK293T cells were transfected with these plasmids by using lipofectamine 2000. After transfection with the plasmids, the HEK293T cells were allowed to stand at 37° C. for 36 hours and then washed with a phosphoric acid buffer (PBS). A culture medium containing CATP (5 μM) was added thereto, which then was allowed to stand for 30 minutes. Thereafter, the culture medium was replaced by a culture medium containing 10% fetal bovine serum (FBS) and the cells then were observed with a fluorescence microscope. With the fluorescence microscope having an excitation wavelength of 436 nm, the cells were observed with a fluorescent filter of 470 to 495 nm. FIG. 5 shows the reaction procedure and FIG. 6 shows the fluorescence microscopic images obtained thereby.

As shown in FIGS. 4 and 6, both fluorescent labeling with FCTP and that with CATP were observed in the PYP-TM expressed cells. On the other hand, neither fluorescent labeling with FCTP nor that with CATP was observed in the cells where PYP-TM was not expressed. These results confirmed that both FCTP and CATP specifically labeled the PYP on the cell membranes. Furthermore, as shown in FIG. 6, fluorescent labeling with CATP was observed in the cells in which MBP-PYP was expressed, while fluorescent labeling with CATP was not observed in the cells in which MBP-PYP was not expressed. These results confirmed that CATP specifically labeled the PYP inside the cultured cell.

### INDUSTRIAL APPLICABILITY

The method for fluorescently labeling a protein of the present invention can be used, for example, for cell imaging.

### SEQUENCE LISTING FREE TEXT

SEQ ID NO: 30: Forward Primer
SEQ ID NO: 31: Reverse Primer

1. A method for fluorescently labeling a protein comprising:
obtaining a fusion protein of a target protein to be labeled and a photoactive yellow protein (PYP) or a PYP-derived protein, and fluorescently labeling the target protein by reacting the fusion protein with a compound represented by Formula (I) below or a salt thereof,
wherein in the compound represented by Formula (I) or the salt thereof, a coumarin skeleton in Formula (A) or a benzene skeleton in Formula (B) is intramolecularly associated with a group X and as a result, fluorescence derived from the group X is suppressed, and
when in the compound represented by Formula (I) or the salt thereof, the coumarin skeleton in Formula (A) or the benzene skeleton in Formula (B) is bound to the PYP or the PYP-derived protein contained in the fusion protein, the coumarin skeleton in Formula (A) or the benzene skeleton in Formula (B) is intramolecularly dissociated from the group X, and thereby fluorescence derived from the group X can be emitted, 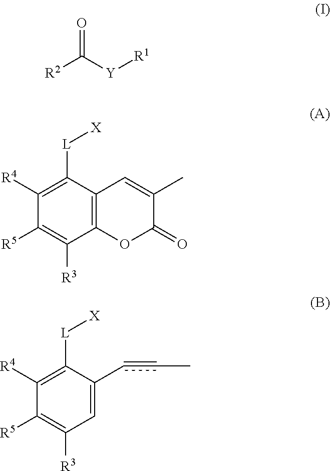
wherein in Formula (I),
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including the coumarin skeleton or a group represented by Formula (B) including the benzene skeleton, wherein in Formula (A) and Formula (B),
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen,
R⁵ is a hydroxy group, an amino group, an amino group that has been mono- or di-substituted with lower alkyl, or a lower alkoxy group, and [symbol]
denotes a double bond or a triple bond.

2. The method for fluorescently labeling a protein according to claim 1, wherein the amino acid sequence of the PYP or PYP-derived protein in the fusion protein is one selected from the group consisting of:
1) amino acid sequences represented by SEQ ID NOS: 1 to 17 of the sequence listing,
2) amino acid sequences represented by SEQ ID NOS: 1 to 17 of the sequence listing in which the 1st to 24th, 25th, 26th, or 27th amino acids have been deleted,
3) amino acid sequences in which one to several amino acids of the amino acid sequences of either 1) or 2) described above have been deleted, substituted, or added, wherein when the fusion protein binds to the coumarin skeleton in Formula (A) or the benzene skeleton in Formula (B), the coumarin skeleton in Formula (A) or the benzene skeleton in Formula (B) is intramolecularly dissociated from the group X in the compound represented by Formula (I), and thereby fluorescence derived from the group X can be emitted, and
4) amino acid sequences having a homology of at least 70% with the amino acid sequences of either 1) or 2) described above, wherein when the fusion protein binds to the coumarin skeleton in Formula (A) or the benzene skeleton in Formula (B), the coumarin skeleton in Formula (A) or the benzene skeleton in Formula (B) is intramolecularly dissociated from the group X in the compound represented by Formula (I), and thereby fluorescence derived from the group X can be emitted.

3. The method for fluorescently labeling a protein according to claim 1, wherein obtaining the fusion protein comprises obtaining a polynucleotide for coding the fusion protein, obtaining a plasmid or vector that can express the fusion protein, expressing the fusion protein in a cell, or isolating the fusion protein thus expressed.

4. A composition to be used for a method according to claim 1, comprising a compound represented by Formula (I) or a salt thereof, 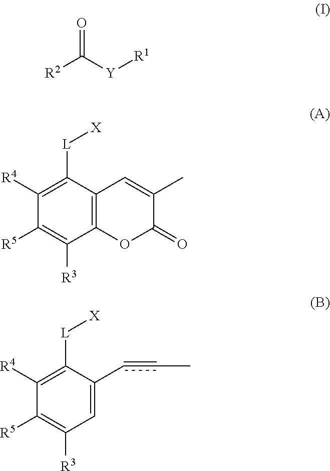
wherein in Formula (I),
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including a coumarin skeleton or a group represented by Formula (B) including a benzene skeleton,
wherein in Formula (A) and Formula (B),
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen,
R⁵ is a hydroxy group, an amino group, an amino group that has been mono- or di-substituted with lower alkyl, or a lower alkoxy group, and [symbol]
denotes a double bond or a triple bond.

5. A plasmid or vector to be used for a method according to claim 1,
wherein the plasmid or vector is one for cloning a polynucleotide for coding a fusion protein of a target protein to be labeled and a PYP or PYP-derived protein or one for expressing the fusion protein, and
the plasmid or vector comprises a base sequence selected from the group consisting of:
1) base sequences represented by SEQ ID NOS: 18 to 29 of the sequence listing,
2) base sequences represented by SEQ ID NOS: 18 to 29 of the sequence listing in which base sequences have been deleted corresponding to deletion of the 1st to 24th, 25th, 26th, or 27th at the N terminus of the amino acid sequences that are coded with the base sequences,
3) base sequences in which one to several bases of the base sequences of either 1) or 2) described above have been deleted, substituted, or added, wherein the base sequences code amino acid sequences in which when the fusion protein binds to the coumarin skeleton in Formula (A) or the benzene skeleton in Formula (B), the coumarin skeleton in Formula (A) or the benzene skeleton in Formula (B) is intramolecularly dissociated from the group X in the compound represented by Formula (I), and thereby fluorescence derived from the group X can be emitted, and
4) base sequences that have a homology of at least 70% with the base sequences of either 1) or 2) described above, wherein the base sequences code amino acid sequences in which when the fusion protein binds to the coumarin skeleton in Formula (A) below or the benzene skeleton in Formula (B) below, the coumarin skeleton in Formula (A) or the benzene skeleton in Formula (B) is intramolecularly dissociated from the group X in the compound represented by Formula (I), and thereby fluorescence derived from the group X can be emitted, 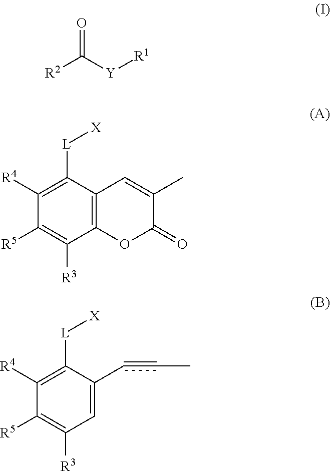
wherein in Formula (I),
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including the coumarin skeleton or a group represented by Formula (B) including the benzene skeleton,
wherein in Formula (A) and Formula (B),
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen, R⁵ is a hydroxy group, an amino group, an amino group that has been mono- or di-substituted with lower alkyl, or a lower alkoxy group, and [symbol]
denotes a double bond or a triple bond.

6. A kit for a method for fluorescently labeling a protein, comprising a compound represented by Formula (I) or a salt thereof, 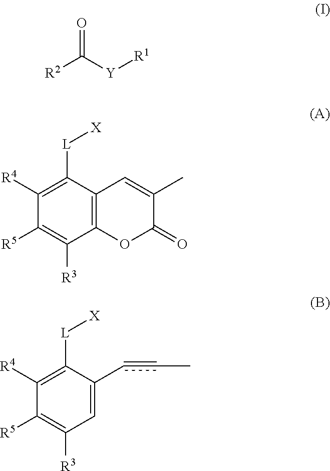
wherein in Formula (I),
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including a coumarin skeleton or a group represented by Formula (B) including a benzene skeleton,
wherein in Formula (A) and Formula (B),
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen,
R⁵ is a hydroxy group, an amino group, an amino group that has been mono- or di-substituted with lower alkyl, or a lower alkoxy group, and [symbol]
denotes a double bond or a triple bond; and
a plasmid or vector according to claim 5.

7. A compound represented by General Formula (I) or a salt thereof, 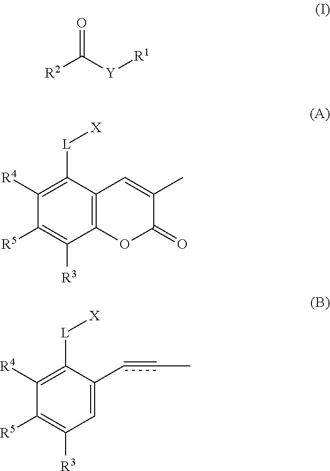
wherein in Formula (I),
Y is an oxygen atom or a sulfur atom,
R¹ is a lower alkyl group, a lower alkyl group substituted with halogen, an aryl group, an aryl group substituted with at least one selected from the group consisting of hydroxy, halogen, and lower alkoxy, a lower aralkyl group, or a lower aralkyl group substituted with at least one selected from the group consisting of hydroxy, halogen, and lower alkoxy, and
R² is a group represented by Formula (A) including a coumarin skeleton or a group represented by Formula (B) including a benzene skeleton,
wherein in Formula (A) and Formula (B),
X is a fluorescent group,
L is a linker for X,
R³ and R⁴ are each independently a hydrogen atom, a halogen atom, a lower alkyl group, a hydroxy group, a lower alkoxy group, or a lower alkyl group substituted with halogen,
R⁵ is a hydroxy group, an amino group, an amino group that has been mono- or di-substituted with lower alkyl, or a lower alkoxy group, and [symbol]
denotes a double bond or a triple bond.

8. The compound or the salt thereof according to claim 7,
wherein
Y is a sulfur atom,
R¹ is an aryl group or an aryl group substituted with at least one selected from the group consisting of hydroxy, halogen, and lower alkoxy, and
R² is a group represented by Formula (A) or Formula (B),
wherein in Formula (A) and Formula (B),
X is a group represented by Formula (i),
L is a group represented by Formula (a), Formula (b), Formula (c), or Formula (d),
R³ and R⁴ each are a hydrogen atom,
R⁵ is hydroxy, and [symbol]
denotes a double bond,
wherein in Formula (a) and Formula (b),
n1, n2, n3, n4, n5, and n6 are each independently an integer of 0 or 1 to 5, and
R¹¹ is a hydrogen atom or a lower alkyl group, and
in Formula (c) and Formula (d),
n1, n2, n3, n4, n5, n6, and n7 are each independently an integer of 0 or 1 to 5, and
R¹¹ and R¹² are each independently a hydrogen atom or a lower alkyl group, [chemical structure]


SEQUENCE LISTING (31 sequences)

SEQ ID NO: 1 - protein, 125 aa, Halorhodospira halophila
MEHVAFGSEDIENTLAKMDDGQLDGLAFGAIQLDGDGNILQYNAAEGDITGRDPKQVIGKNFFKDVAPCTDSPEFYGKFKEGVASGNLNTMFEYTFDYQMTPTKVKVHMKKALSGDSYWVFVKRV

SEQ ID NO: 2 - protein, 124 aa, Rhodobacter capsulatus
MEIIPFGTNDIDNILAREPARAESLPFGAVLLDRMGRIAKYNKAEGLIAGRDPSTVIGRDFFNEIAPCAKGKRFHGEFLKFNRTGQANVMLDYKFNYKGAEVAVKIHLKSQPDGQFCWLFVKRA

SEQ ID NO: 3 - protein, 124 aa, Rhodobacter sphaeroides
MEIIPFGSADLDNILAREPQRAEYLPFGAVLLDRTGTILKYNRAEGGIANRNPADVIGKNFFNEIAPCAKGKRFHGEFLRFHQTGQVNVMFDYKFAYKGANVGVKIHMKSQPDGQSCWLFVKRV

SEQ ID NO: 4 - protein, 125 aa, Rhodothalassium salexigens
MEMIKFGQDDIENAMADMGDAQIDDLAFGAIQLDETGTILAYNAAEGELTGRSPQDVIGKNFFKDIAPCTDTEEFGGRFREGVANGDLNAMFEYVFDYQMQPTKVKVHMKRAITGDSYWIFVKRV

SEQ ID NO: 5 - protein, 135 aa, Stigmatella aurantiaca
MRHGILEAESLTEDRLGQLSPEEFDALPFGAIKLDAEGRVLIYNAAESAFSRRKPVSVLGRRFFEEVAPCTNVASFRGRFDTLVERGHGTESFDFQFRFRWGTRNVRIRLMVLGDGSRWVFVTAVLTALIPLGEG

SEQ ID NO: 6 - protein, 131 aa, Sorangium cellulosum
MGSEERSTAGEFEFDIGVFNLDERGLDAQPFGIIRLDREGTVLSYNLYEERQARRNRQDVIGKNFFTDIAPCSRVKAFHGRFLAGVEQRELKATFGFVFHFPHKTRHVDVSLFYKAAARQQDDAVWVFIRG

SEQ ID NO: 7 - protein, 123 aa, Leptospira biflexa serovar Patoc
MSKFIDPNILGKLGTLAQAEADGYPFGIVKVDESGKILLYNKYESELANVPIQTAVGKNFFTEVAICTNNRIFYGRFKEGMISGDLDIAFNYVFTYKMKPTNVVIHLYHDKGTNSNWIFVKLR

SEQ ID NO: 8 - protein, 130 aa, Leptothrix cholodnii
MNEALPLVFDQPDLAACIGTLSEAQLDGLGFGVIGFDAQGVVRVYNAFESKYAGLSPQRVLGHPLFTVVAPCMNNFMVAQRFEDAAASAASLDATIDYVLTLRMRPVKVKLRLLAAPATALRYVLVQRPA

SEQ ID NO: 9 - protein, 123 aa, Burkholderia phytofirmans
MDNEFESVRIAELAMLDADRLDGVPFGVIGFTSDALVTVYNATESKNAGLRPKMVLGKHFFGEVAPCMNNFMVAQRFEDEDVLDDIVPYVLTLRMRPTPVRLRLLKATDCATRFVLIERRATN

SEQ ID NO: 10 - protein, 156 aa, Salinibacter ruber
MADSQNPYSYLREDDPDSAPGDSGDADEPEPPATDLAFDDEGVGEELRHVDEDELNAAPFGIIQIDDAGVVQFYNRYESNLSGIDPADAVGANFFTELAPCSNNPLFFGRFKDGVREGGLDEYFTYTFTYQMRPTLVDVRLYRDEAENNWILIQKR

SEQ ID NO: 11 - protein, 125 aa, Idiomarina loihiensis
MEIVQFGSDDIENTLSKMSDDKLNDIAFGAIQLDASGKIIQYNAAEGDITGRDPGAVVGKNFFNEVAPCTNSPEFKGRFDEGVKNGNLNTMFEYVFDYEMQPTKVKVHMKKALTGDTYWVFVKRL

SEQ ID NO: 12 - protein, 130 aa, Rhodospirillum centenum
MPDRTTDDFGPFTEQIRGTIDGMGTAEFDALPVGAIQVDGSGVIHRYNRTESRLSGRIPERVIGRNFFTEVAPCTNIPAFSGRFMDGVTSGTLDARFDFVFDFQMAPVRVQIRMQNAGVPDRYWIFVRKL

SEQ ID NO: 13 - protein, 125 aa, Thermochromatium tepidum
MNFEDAIDIHAPRRLDALTPDELNRLPFGAIRVDAEGRILFYSRALVDLANRQVDSVLGRNFFSEIAPCTVVPEFYGRFRQGVLTGQLHTTFEFVFDFDMQPVQVRIAMHTSERPGEFWILVQPL

SEQ ID NO: 14 - protein, 126 aa, Rhodopseudomonas palustris
MNTVDFHDSDLARTIEQLAPEQIDALPFGVIKLDGNGIVTVFNRTEAIESGYKSRPALGLDFFLQVAPCMGQPEFRGRIEQARQLGRVDIELGWVGDFSDINRSLQVRIQSASDGSLWIFNLRDHA

SEQ ID NO: 15 - protein, 146 aa, Stigmatella aurantiaca PYP2
MAPPSTSLALSNAPPPKTTPTDDLLRQVETLSASELDALPFGLIQLDRTGRILKFNQTEAKLARINRERQLGRNFFDDVAPCTKVREFYGRFLNGLSQRSLYETFGFIFKFDHGWRNVAITMFYSEKTDSVWVLISQTSVTPPPAR

SEQ ID NO: 16 - protein, 130 aa, Halorhodospira halophila PYP2
MGTLIFGRQDLENRLAAMTPEEIDDLPFGVIQIDQHGRILLYNATEGAITGRDPEAMIGRDFFNDVAPCGHTEAFYGRFQEGVRHGDLNEIFDYTFDYRMAPTKVRVHMKRALSGDTYWIFVKRISAPAA

SEQ ID NO: 17 - protein, 125 aa, Halochromatium salexigens
MNIVHFGSDDIENSLANMSDQDLNQLAFGAIQLDASGKVLQYNAAEEGITGRDPKSVIGKNFFEDVAPCTKSQEFQGRFKEGVANGNLATMFEYVFDYQMKPTKVKVHMKKALVDDSYWIFVKRL

SEQ ID NO: 18 - DNA, 378 bp, Halorhodospira halophila
atggaacacgtagccttcggtagcgaggacatcgagaacaccctcgccaagatggacgacggccagctcgacggcctggccttcggcgccatccagctcgacggcgacggcaacatccttcagtacaacgccgcggagggcgacatcaccggccgcgacccgaagcaggtcatcggcaagaacttcttcaaggacgtggccccgtgcactgacagcccggagttctacggcaagttcaaggaaggggtggcctcgggcaacctgaacacgatgttcgagtacaccttcgattaccaaatgacgcccacgaaggtgaaggtgcacatgaagaaggccctctccggcgacagctactgggtcttcgtcaagcgcgtctag

SEQ ID NO: 19 - DNA, 375 bp, Rhodobacter capsulatus
atggaaatcattccgttcgggacgaacgacatcgacaacatcctggcgcgcgagcccgcgcgtgcggaaagcctgccgttcggcgccgtgcttctcgaccgcatggggcggatcgccaaatacaacaaggccgaggggctgatcgcgggccgcgatccctcgacggtgatcggccgcgatttcttcaacgagatcgcgccctgcgccaagggcaagcggttccacggggaattcctgaaattcaaccgcaccggccaggccaatgtgatgctggactacaagttcaattacaagggcgccgaagtggcggtgaagatccacctcaagtcccaacccgacggccagttctgctggctcttcgtgaagcgggcctga

SEQ ID NO: 20 - DNA, 375 bp, Rhodobacter sphaeroides
atggaaatcattccctttggcagcgccgacctcgacaacatccttgcccgcgagccgcagcgggccgagtatctccccttcggcgccgtcctgctggaccgcaccggcacgatcctgaaatacaacagggccgagggtggcatcgccaaccgcaatccggccgacgtgatcggaaagaacttcttcaacgagatcgcgccctgcgccaagggcaagcgctttcacggcgagttcctgcgctttcaccagaccggacaggtcaatgtgatgttcgattacaaattcgcctacaagggcgccaatgtcggcgtgaagatccacatgaaatcgcagcccgacggccagagctgctggctcttcgtcaaacgggtctga

SEQ ID NO: 21 - DNA, 378 bp, Rhodothalassium salexigens
atggaaatgatcaaattcggccaggacgacatcgagaacgccatggcggatatgggcgacgcgcagatcgacgacctggcttttggcgccattcaactggacgagaccggcacgatcctggcctataacgcggccgagggcgaactaaccggccgcagtccccaagacgtgatcggcaagaacttcttcaaggacatagcgccgtgcaccgacaccgaggaattcggcggccggttccgcgaaggggtggccaatggcgacctgaacgcgatgttcgaatatgtcttcgactatcagatgcagccgaccaaggtgaaggtgcacatgaagcgcgctatcaccggcgacagctactggatcttcgtcaagcgtgtctga

SEQ ID NO: 22 - DNA, 408 bp, Stigmatella aurantiaca
atgcggcatgggattctggaagcggagtcgctgacagaggatcggctgggccagctgtcgcccgaggagtttgatgcgttgccgtttggcgccatcaagctggacgcggagggacgggtgctcatctacaacgcggcggagtccgcgttctcccggcgcaagcccgtgtccgtgctcggccgccgcttcttcgaggaggttgccccttgtaccaacgtggccagcttccgggggcgtttcgacacgctcgtcgagcgagggcatggcacggagagcttcgacttccagttccgattccgctggggcacacgcaacgtgcgcatccggctgatggtgctgggggacggttcccgctgggtcttcgtgacggcggtgctgacggcgctgatccccctcggcgagggctga

SEQ ID NO: 23 - DNA, 396 bp, Sorangium cellulosum
atgggttcggaggagcggtccacagccggtgagttcgagttcgatatcggggtgttcaacctggatgaacgcggcctggacgcgcagcccttcgggatcatccggctggatcgcgagggcacggtgctctcctacaacctctacgaggaacgtcaggcgcgccggaatcggcaggacgtgatagggaagaacttcttcaccgacatcgcgccctgcagccgggtgaaggcgttccacggccgcttcctggccggggtcgagcagcgcgagctcaaggccaccttcggcttcgtgttccacttccctcacaagacacggcacgtcgacgtctcgctgttctacaaagcggccgcgcgacagcaggacgatgcggtctgggtgttcatccgaggttga

SEQ ID NO: 24 - DNA, 372 bp, Leptospira biflexa serovar Patoc
atgagcaaatttatagacccaaatattttaggaaaactcggtaccttggcacaagcggaagctgacggatacccgtttggaattgtcaaagtagacgagtcgggtaaaattttattgtataacaaatacgaatccgaacttgccaatgttcccatccaaacagccgtagggaaaaacttttttacggaagtggctatttgtacgaacaaccgaatcttctatggaaggtttaaagaagggatgatttcgggagatttggacatcgcattcaattatgtattcacttacaaaatgaaaccaaccaatgttgtgatccatctctaccatgacaaagggaccaattccaattggatttttgtcaaactcagataa

SEQ ID NO: 25 - DNA, 393 bp, Leptothrix cholodnii
atgaatgaagcgctgccgctggtgttcgaccagcctgatctggctgcctgcatcggcacgctgagcgaggcgcagctcgatggcctcggcttcggtgtgatcggcttcgatgcgcagggtgtggtgcgtgtctacaacgccttcgagtcgaaatatgccgggctgtcgccgcagcgggtgttgggccatccgctgttcaccgtggtggcaccgtgcatgaacaacttcatggtcgcccagcgcttcgaagacgccgccgccagcgccgccagcctcgacgccaccatcgactacgtgctgacgctgcgcatgcgcccggtcaaggtcaagctccgattgctggccgcgcctgccacggccttgcgctacgtgctcgtgcaacgcccggcctga

SEQ ID NO: 26 - DNA, 372 bp, Burkholderia phytofirmans
gtggacaacgaattcgaaagtgtgcggatagcagaactggccatgctggacgcggaccggttagacggggtgccatttggcgtcatcggatttacctcggatgcacttgtcacggtctataacgctactgaatcgaagaacgccggattgcggccaaagatggtgttagggaagcatttcttcggagaggtcgcaccgtgcatgaacaatttcatggtggcgcagcgcttcgaagatgaggacgtacttgacgacatcgttccctacgttttgaccttgcgcatgcgtccgacgccggtgcggctcaggctcctgaaagccacggattgcgcgacacggtttgtcctgattgagcgaagggcgacaaattga

SEQ ID NO: 27 - DNA, 471 bp, Salinibacter ruber
atggctgactctcagaatccgtactcgtacctgcgtgaagacgacccggactccgcccccggcgactcgggcgacgccgacgagcctgagccgccggcaacggacctcgcgttcgacgacgagggggtgggggaggagctgcgtcacgtcgacgaggacgagctaaatgcggccccgttcggcatcattcagattgacgacgcgggggtcgtgcagttctacaaccgctacgagtcgaacctaagcggcatcgatccggcagacgccgtcggggccaacttcttcacggagctggcgccgtgcagcaacaacccgctgttcttcggccgcttcaaggacggggttcgggagggggggctcgacgagtacttcacgtacacgttcacctaccagatgcgccccaccctcgtcgacgtgcggctctaccgcgacgaggcggagaacaactggatcctgattcagaagcggtga

SEQ ID NO: 28 - DNA, 378 bp, Idiomarina loihiensis
atggagattgttcaattcggttctgatgatatcgaaaacactttatctaaaatgtcagatgacaagttaaacgacattgcttttggcgctattcagttagacgctagcggtaaaattattcaatacaacgccgccgaaggcgacattaccggtcgtgatccgggcgctgtcgttggcaaaaacttttttaacgaagtggcaccatgcaccaacagccctgagtttaaaggacgcttcgacgaaggcgttaaaaacggcaatttaaacaccatgtttgaatatgtttttgactacgaaatgcaaccgaccaaagttaaagttcatatgaaaaaagctttaaccggcgacacctactgggtattcgttaagcgactataa

SEQ ID NO: 29 - DNA, 390 bp, Rhodospirillum centenum
gtgccggaccggaccaccgacgatttcggccccttcaccgagcagatccgcggcaccatcgacgggatggggacggccgagttcgatgccttgccggtcggcgccatccaggtggacggcagcggggtcatccaccgttacaacaggacggaaagccggctcagcggccgcatccccgaacgggtcatcgggcgcaacttcttcaccgaggtggcgccctgcacgaacatcccggccttcagcggccgcttcatggacggtgtgacctccggcacgctggatgcccgcttcgacttcgtcttcgatttccagatggcgccggtgcgggtgcagatccgcatgcagaacgcgggcgtgcccgaccgctactggatcttcgtgcgcaagctg

SEQ ID NO: 30 - DNA, 31 bp, Artificial Sequence
  Forward Primer
cggagatatacatatgagaggatcgcatcac

SEQ ID NO: 31 - DNA, 21 bp, Artificial Sequence
  Reverse Primer
gctaattaagcttctagacgc